(19) United States
(12) Patent Application Publication
(10) Pub. No.: US 2017/0319453 A1
(43) Pub. Date: Nov. 9, 2017

(54) CLEANSING COMPOSITION

(71) Applicant: L'OREAL, Paris (FR)

(72) Inventor: Shuzo ANDO, Kawasaki-shi, Kanagawa (JP)

(21) Appl. No.: 15/533,828

(22) PCT Filed: Nov. 27, 2015
(86) PCT No.: PCT/JP2015/084079

§ 371 Date: Jun. 7, 2017

(30) Foreign Application Priority Data
Dec. 12, 2014 (JP) 2014-251421

(51) Int. Cl. A61K 8/46; A61Q 5/02; A61K 8/60; A61K 8/37; A61K 8/04; A61K 8/44; A61Q 19/10; A61K 8/73

(52) U.S. Cl. CPC A61K 8/466; A61Q 19/10; A61Q 5/02; A61K 8/602; A61K 8/731; A61K 8/046; A61K 8/463; A61K 8/442; A61K 8/375; A61K 2800/52

(57) ABSTRACT

The present invention relates to a composition comprising: (a) at least one linear olefin sulfonate; (b) at least one glycerin fatty acid ester; and (c) at least one amphoteric surfactant. The composition according to the present invention can have no irritation or reduced irritation, and superior foam quality, and may be stable, in particular stable over time and/or under elevated temperature. The composition according to the present invention may preferably be a cosmetic composition, and more preferably a cleansing composition, such as a shampoo.

### TECHNICAL FIELD

The present invention relates to a composition, preferably a cosmetic composition, and more preferably a cleansing composition, which has no irritation or reduced irritation, and superior foam quality.

### BACKGROUND ART

Compositions for cleansing hair, in particular human hair, which comprises surfactants as an active ingredient are widely known. Such compositions commonly comprise one or more surfactants selected from conventional surfactants especially of the anionic, nonionic and/or amphoteric type, but more particularly of the anionic type. A composition for cleansing hair is applied to wet hair and the foam generated by massaging or rubbing with the hands allows, after rinsing with water, the removal of the various types of dirt initially present on the hair.

Conventionally, Sodium Laureth Ether Sulfate (SLES) has been used as a surfactant in hair cleansing compositions such as a shampoo. It is known that SLES exhibits good detergent properties. However, SLES tends to make human hair and the scalp rough because of its strong surface-active properties.

Furthermore, oxyalkylene units, in particular oxyethylene units, in SLES may produce oxylane. Oxylane is a kind of poisonous material which can induce cancer. So, in order to avoid such kinds of risks, sulfate free anionic surfactant has gradually become the focus of attention. In fact, some sulfate-free shampoos, which are said to be gentle to the hair and scalp, have been marketed.

Thus, a shampoo composition which is gentle to the hair and human health as well as being moderate and at least comparable, in terms of foam quality and cosmetic properties, with SLES shampoo compositions has been desired.

### DISCLOSURE OF INVENTION

An objective of the present invention is to provide a composition, preferably a cosmetic composition, and more preferably a cleansing composition, which has no irritation or reduced irritation, and superior foam quality. It is preferable that the composition is stable, in particular stable over time and/or under elevated temperature.

The above objective of the present invention can be achieved by a composition comprising:

(a) at least one linear olefin sulfonate;

(b) at least one glycerin fatty acid ester; and

(c) at least one amphoteric surfactant.

It is preferable that the (a) linear olefin sulfonate be selected from linear C₁₂-C₁₈ olefin sulfonates.

The amount of the (a) linear olefin sulfonate may range from 1 to 20% by weight, preferably from 3 to 15% by weight, and more preferably from 5 to 10% by weight, relative to the total weight of the composition.

It is preferable that the (b) glycerin fatty acid ester be a monoester.

The (b) glycerin fatty acid ester may be a monoester of glycerin and a C₈-C₁₈ fatty acid, preferably C₈-₁₄ fatty acid, and more preferably C₈-C₁₂ fatty acid.

The (b) glycerin fatty acid ester may be selected from the group consisting of glyceryl caprylate, glyceryl caprate, glyceryl laurate, glyceryl myristate, glyceryl stearate, glyceryl isostearate and a mixture thereof.

The amount of the (b) glycerin fatty acid ester may range from 0.01 to 10% by weight, preferably from 0.05 to 5% by weight, and more preferably from 0.1 to 1% by weight, relative to the total weight of the composition.

It is preferable that the (c) amphoteric surfactant be selected from betaine-type surfactants.

The amount of the (c) amphoteric surfactant may range from 0.01 to 10% by weight, preferably from 0.05 to 7% by weight, and more preferably from 0.1 to 5% by weight, relative to the total weight of the composition.

The composition according to the present invention may further comprise (d) at least one nonionic surfactant, preferably alkylpolyglucoside.

The amount of the (d) nonionic surfactant may range from 0.01 to 10% by weight, preferably from 0.05 to 7% by weight, and more preferably from 0.1 to 5% by weight, relative to the total weight of the composition.

The composition according to the present invention may further comprise (e) at least one cationic polymer.

The composition according to the present invention may comprise 1% by weight or less, preferably 0.1% by weight or less, and more preferably 0.01% by weight or less of alkylsulfates or alkylethersulfates, relative to the total weight of the composition.

It is preferable that the composition according to the present invention be intended for a cosmetic composition, more preferably a cleansing composition, and even more preferably a cleansing composition for keratin fibers.

The present invention also relates to a use of the composition according to the present invention for the manufacture of a cleansing composition, preferably a shampoo, with no irritation or reduced irritation to the scalp.

The present invention also relates to a cosmetic process for treating the skin, the hair, and/or the scalp, comprising the step of applying the composition according to the present invention to the skin, the hair, and/or the scalp.

### BEST MODE FOR CARRYING OUT THE INVENTION

After diligent research, the inventors have discovered that it is possible to provide a composition which has superior foam quality by a specific combination of specific ingredients, even without SLES.

The composition can have no irritation or reduced irritation, because it no longer needs SLES. The composition can also be stable, depending on the amount of the (c) amphoteric surfactant in the composition.

Thus, one aspect of the present invention is a composition comprising:

(a) at least one linear olefin sulfonate;

(b) at least one glycerin fatty acid ester; and

(c) at least one amphoteric surfactant.

The composition according to the present invention has no irritation or reduced irritation, and has superior foam quality represented by the volume of foam. Therefore, the composition according to the present invention can be a cosmetic composition, preferably a cleansing composition, and more preferably a shampoo. In addition, the composition according to the present invention can be stable, in particular stable over time and/or under elevated temperature.

Hereinafter, the composition according to the present invention will be explained in a more detailed manner.

### Linear Olefin Sulfonate

The composition according to the present invention comprises at least one linear olefin sulfonate. A single type of linear olefin sulfonate may be used, but two or more different types of linear olefin sulfonate may be used in combination.

The linear olefin sulfonate has a linear, olefin moiety and a sulfonate moiety, and can function as an anionic surfactant. The linear olefin moiety may include at least one carbon-carbon double bond. The olefin moiety may directly bond to the sulfonate moiety, without any spacer such as an aromatic moiety, e.g., a phenylene group.

The linear olefin sulfonate is preferably selected from linear α-olefin sulfonates.

The α-olefin sulfonate may be represented by the following formula:

R—CH═CH—(CH₂)ₙ—SO₃M

wherein

R denotes an aliphatic moiety, n denotes an integer from 0 to 10, preferably 1 to 4, and more preferably 1, and M denotes a monovalent cation. R may preferably be a linear C₈-C₃₀ alkyl or alkenyl group, more preferably a linear C₁₀-C₂₀ alkyl or alkenyl group, and even more preferably a linear C₁₂-C₁₈ alkyl or alkenyl group, which may include at least one heteroatom such as O and N. M may preferably be an alkali metal cation, such as Na⁺ and Ka⁺.

It is preferable that the (a) linear olefin sulfonate be selected from linear C₁₂-C₁₈ olefin sulfonates, more preferably liner C₁₄-C₁₆ olefin sulfonates, and even more preferably linear C₁₄-C₁₆ α-olefin sulfonates.

As examples of the (a) linear olefin sulfonate, mention may be made of sodium C₁₄-C₁₆ olefin sulfonate such as Bio-Terge AS-40 sold by Stepan or Hostapur OSB sold by Clariant, sodium C₁₆-C₁₈ olefin sulfonate, and sodium C₁₄-C₁₈ olefin sulfonate such as Colonial AOS-40 sold by Colonial Chemical Inc. Sodium C₁₄-C₁₆ olefin sulfonate is preferable as the (a) linear olefin sulfonate.

The α-olefin sulfonate may be present together with a hydroxyalkylsulfonate represented by the following formula:

R—CH(OH)CH₂(CH₂)ₙ—SO₃M or R—CH₂CH(OH)(CH₂)ₙ—SO₃M

wherein R, n and M have the same meanings as above.

The amount of the (a) linear olefin sulfonate is not limited, but may range from 1 to 20% by weight, preferably from 3 to 15% by weight, and more preferably from 5 to 10% by weight, relative to the total weight of the composition.

### Glycerin Fatty Acid Ester

The composition according to the present invention comprises at least one glycerin fatty acid ester. A single type of glycerin fatty acid ester may be used, but two or more different types of glycerin fatty acid ester may be used in combination.

The (b) glycerin fatty acid ester is an ester of glycerin and fatty acid(s). It is preferable that the (b) glycerin fatty acid ester be a monoester, wherein one molecule of a fatty acid is ester bonded to any hydroxyl group of glycerin. The ester is otherwise called monoacylglycerin or monoglyceride. Examples of the fatty acids include straight-chain fatty acids, branched-chain fatty acids, saturated fatty acids, unsaturated fatty acids and hydroxy fatty acids. It is preferable that the (b) glycerin fatty acid ester is non polyoxyalkylenated.

Examples of the (b) glycerin mono fatty acid esters include glyceryl caprylate, glyceryl caprate, glyceryl laurate, glyceryl myristate, glyceryl stearate, glyceryl linoleate, glyceryl oleate, glyceryl isostearate, glyceryl behenate, glyceryl erucate, glyceryl cocofatty acid ester, glyceryl ricinoleate, glyceryl hydroxystearate, wheat germ oil fatty acid monoglyceride, safflower oil fatty acid monoglyceride, hydrogenated soybean fatty acid monoglyceride, saturated fatty acid monoglyceride, cotton seed oil fatty acid monoglyceride, tallow fatty acid monoglyceride and lanolin fatty acid monoglyceride.

The (b) glycerin fatty acid ester may preferably be a monoester of glycerin and C₈-C₁₈ fatty acid, preferably C₈-C₁₄ fatty acid, and more preferably C₈-C₁₂ fatty acid. Thus, the (b) glycerin fatty acid ester may preferably be selected from the group consisting of glyceryl caprylate, glyceryl caprate, glyceryl laurate, glyceryl myristate, glyceryl stearate, glyceryl isostearate and mixtures thereof.

As the (b) glycerin mono fatty acid ester, a monoester of glycerin and a linear fatty acid is preferable, and a monoester of glycerin and a linear C₈-C₁₀ fatty acid are more preferable. Thus, as the (b) glycerin mono fatty acid ester, glyceryl caprylate, glyceryl caprate, and a mixture thereof are preferable, and glyceryl caprylate is more preferable.

The amount of the (b) glycerin fatty acid ester is not limited, but may range from 0.01 to 10% by weight, preferably from 0.05 to 5% by weight, and more preferably from 0.1 to 1% by weight, relative to the total weight of the composition.

### Amphoteric Surfactant

The composition according to the present invention comprises at least one amphoteric surfactant. A single type of amphoteric surfactant may be used, but two or more different types of amphoteric surfactant may be used in combination.

The amphoteric or zwitterionic surfactants can be, for example (non-limiting list), amine derivatives such as aliphatic secondary or tertiary amine, and optionally quaternized amine derivatives, in which the aliphatic radical is a linear or branched chain comprising 8 to 22 carbon atoms and containing at least one water-solubilizing anionic group (for example, carboxylate, sulfonate, sulfate, phosphate or phosphonate).

The (c) amphoteric surfactant may preferably be selected from the group consisting of betaines and amidoaminecarboxylated derivatives.

It is preferable that the (c) amphoteric surfactant be selected from betaine-type surfactants.

The betaine-type amphoteric surfactant is preferably selected from the group consisting of alkylbetaines, alkylamidoalkylbetaines, sulfobetaines, phosphobetaines, and alkylamidoalkylsulfobetaines, in particular, (C₈-C₂₄)alkylbetaines, (C₈-C₂₄)alkylamido(C₁-C₈)alkylbetaines, sulfobetaines, and (C₈-C₂₄)alkylamido(C₁-C₈)alkylsulfobetaines. In one embodiment, the amphoteric surfactants of betaine type are chosen from (C₈-C₂₄)alkylbetaines, (C₈-C₂₄)alkylamido(C₁-C₈)alkylsulfobetaines, sulfobetaines, and phosphobetaines.

Non-limiting examples that may be mentioned include the compounds classified in the CTFA dictionary, 9th edition, 2002, under the names cocobetaine, laurylbetaine, cetylbetaine, coco/oleamidopropylbetaine, cocamidopropylbetaine, palmitamidopropylbetaine, stearamidopropylbetaine, cocamidoethylbetaine, cocamidopropylhydroxysultaine, oleamidopropylhydroxysultaine, cocohydroxysultaine, laurylhydroxysultaine, and cocosultaine, alone or as mixtures.

The betaine-type amphoteric surfactant is preferably an alkylbetaine and an alkylamidoalkylbetaine, in particular cocobetaine and cocamidopropylbetaine.

Among the amidoaminecarboxylated derivatives, mention may be made of the products sold under the name Miranol, as described in U.S. Pat. Nos. 2,528,378 and 2,781,354 and classified in the CTFA dictionary, 3rd edition, 1982 (the disclosures of which are incorporated herein by reference), under the names Amphocarboxyglycinates and Amphocarboxypropionates, with the respective structures:

R₁—CONHCH₂CH₂—N⁺(R₂)(R₃)(CH₂COO⁻)

in which:

R₁ denotes an alkyl radical of an acid R₁—COOH present in hydrolysed coconut oil, a heptyl, nonyl or undecyl radical,

R₂ denotes a beta-hydroxyethyl group, and

R₃ denotes a carboxymethyl group; and

R₁′—CONHCH₂CH₂—N(B)(C)

in which:

B represents —CH₂CH₂OX′,

C represents —(CH₂)_{z}—Y′, with z=1 or 2,

X′ denotes a —CH₂CH₂—COOH group, —CH₂—COOZ′, —CH₂CH₂—COOH, —CH₂CH₂—COOZ′ or a hydrogen atom,

Y′ denotes —COOH, —COOZ′, —CH₂—CHOH—SO₃Z′ or a —CH₂—CHOH—SO₃H radical,

Z′ represents an ion of an alkaline or alkaline earth metal such as sodium, an ammonium ion or an ion issued from an organic amine, and

R₁′ denotes an alkyl radical of an acid R₁′—COOH present in coconut oil or in hydrolysed linseed oil, an alkyl radical, such as a C₇, C₉, C₁₁ or C₁₃ alkyl radical, a C₁₇ alkyl radical and its iso form, or an unsaturated C₁₇ radical.

In one embodiment, the amphoteric surfactant may be selected from (C₈-C₂₄)-alkyl amphomonoacetates, (C₈-C₂₄)alkyl amphodiacetates, (C₈-C₂₄)alkyl amphomonopropionates, and (C₈-C₂₄)alkyl amphodipropionates

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphopropionate, Disodium Caprylamphodipropionate, Disodium Caprylamphodipropionate, Lauroamphodipropionic acid and Cocoamphodipropionic acid.

By way of example, mention may be made of the cocoamphodiacetate sold under the trade name Miranol® C2M concentrate by the company Rhodia Chimie.

The amount of the (c) amphoteric surfactant is not limited, but may range from 0.01 to 10% by weight, preferably from 0.05 to 7% by weight, and more preferably from 0.1 to 5% by weight, relative to the total weight of the composition. In terms of stability of the composition according to the present invention, it is preferable that the amount of the (c) amphoteric surfactant range from 0.05 to 7% by weight, more preferably from 0.1 to 5% by weight, even more preferably from 1 to 3% by weight, relative to the total weight of the composition.

### Nonionic Surfactant

The composition according to the present invention may comprise at least one nonionic surfactant, different from compound (b). A single type of nonionic surfactant may be used, but two or more different types of nonionic surfactant may be used in combination.

The nonionic surfactants are compounds well known in themselves (see, e.g., in this regard, “Handbook of Surfactants” by M. R. Porter, Blackie & Son publishers (Glasgow and London), 1991, pp. 116-178). Thus, they can, for example, be chosen from alcohols, alpha-diols, alkylphenols and esters of fatty acids, these compounds being ethoxylated, propoxylated or glycerolated and having at least one fatty chain comprising, for example, from 8 to 30 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils of plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives; amine oxides such as (C₁₀-C₁₄)alkylamine oxides or N—(C₁₀-C₁₄)acylaminopropylmorpholine oxides; silicone surfactants; and mixtures thereof.

The (d) nonionic surfactants may preferably be chosen from monooxyalkylenated, polyoxyalkylenated, monoglycerolated or polyglycerolated nonionic surfactants. The oxyalkylene units are more particularly oxyethylene or oxypropylene units, or a combination thereof, and are preferably oxyethylene units.

Examples of monooxyalkylenated or polyoxyalkylenated nonionic surfactants that may be mentioned include:

monooxyalkylenated or polyoxyalkylenated (C₈-C₂₄)alkylphenols, saturated or unsaturated, linear or branched, monooxyalkylenated or polyoxyalkylenated C₈-C₃₀ alcohols,

saturated or unsaturated, linear or branched, monooxyalkylenated or polyoxyalkylenated C₈-C₃₀ amides,

esters of saturated or unsaturated, linear or branched, C₈-C₃₀ acids and of polyalkylene glycols, monooxyalkylenated or polyoxyalkylenated esters of saturated or unsaturated, linear or branched, C₈-C₃₀ acids and of sorbitol,

saturated or unsaturated, monooxyalkylenated or polyoxyalkylenated plant oils,

condensates of ethylene oxide and/or of propylene oxide, inter alia, alone or as mixtures.

The surfactants preferably contain a number of moles of ethylene oxide and/or of propylene oxide of between 1 and 100 and most preferably between 2 and 50. Advantageously, the nonionic surfactants do not comprise any oxypropylene units.

According to one of the embodiments of the present invention, the polyoxyalkylenated nonionic surfactants are chosen from polyoxyethylenated fatty alcohol (polyethylene glycol ether of fatty alcohol) and polyoxyethylenated fatty ester (polyethylene glycol ester of fatty acid).

Examples of polyoxyethylenated fatty alcohol (or C₈-C₃₀ alcohols) that may be mentioned include the adducts of ethylene oxide with lauryl alcohol, especially those containing from 9 to 50 oxyethylene units and more particularly those containing from 10 to 12 oxyethylene units (Laureth-10 to Laureth-12, as the CTFA names); the adducts of ethylene oxide with behenyl alcohol, especially those containing from 9 to 50 oxyethylene units (Beheneth-9 to Beheneth-50, as the CTFA names); the adducts of ethylene oxide with cetearyl alcohol (mixture of cetyl alcohol and stearyl alcohol), especially those containing from 10 to 30 oxyethylene units (Ceteareth-10 to Ceteareth-30, as the CTFA names); the adducts of ethylene oxide with cetyl alcohol, especially those containing from 10 to 30 oxyethylene units (Ceteth-10 to Ceteth-30, as the CTFA names);

the adducts of ethylene oxide with stearyl alcohol, especially those containing from 10 to 30 oxyethylene units (Steareth-10 to Steareth-30, as the CTFA names); the adducts of ethylene oxide with isostearyl alcohol, especially those containing from 10 to 50 oxyethylene units (Isosteareth-10 to Isosteareth-50, as the CTFA names); and mixtures thereof.

As examples of monoglycerolated or polyglycerolated nonionic surfactants, monoglycerolated or polyglycerolated C₈-C₄₀ alcohols are preferably used.

In particular, the monoglycerolated or polyglycerolated C₈-C₄₀ alcohols correspond to the following formula:

RO—[CH₂—CH(CH₂OH)—O]ₘ—H or RO—[CH(CH₂OH)—CH₂O]ₘ—H

in which R represents a linear or branched C₈-C₄₀ and preferably C₈-C₃₀ alkyl or alkenyl radical, and m represents a number ranging from 1 to 30 and preferably from 1.5 to 10.

As examples of compounds that are suitable in the context of the present invention, mention may be made of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

The alcohol may represent a mixture of alcohols in the same way that the value of m represents a statistical value, which means that, in a commercial product, several species of polyglycerolated fatty alcohol may coexist in the form of a mixture.

Among the monoglycerolated or polyglycerolated alcohols, it is preferable to use the C₈/C₁₀ alcohol containing 1 mol of glycerol, the C₁₀/C₁₂ alcohol containing 1 mol of glycerol and the C₁₂ alcohol containing 1.5 mol of glycerol.

The polyglycerolated C₈-C₄₀ fatty esters (i.e. polyglyceryl C₈₋₄₀ fatty acid ester) may preferably be chosen from the mono, di and tri esters of saturated or unsaturated acid, preferably saturated acid, including 8 to 40 carbon atoms, preferably 10 to 30 carbon atoms, such as lauric acid, oleic acid, stearic acid, isostearic acid, capric acid, caprylic acid, and myristic acid.

The polyglycerolated C₈-C₄₀ fatty esters may be selected from the group consisting of PG2 caprylate, PG2 sesquicaprylate, PG2 dicaprylate, PG2 tricaprylate, PG2 caprate, PG2 sesquicaprate, PG2 dicaprate, PG2 tricaprate, PG2 laurate, PG2 sesquilaurate, PG2 dilaurate, PG2 trilaurate, PG2 myristate, PG2 sesquimyristate, PG2 dimyristate, PG2 trimyristate, PG2 stearate, PG2 sesquistearate, PG2 distearate, PG2 tristearate, PG2 isostearate, PG2 sesquiisostearate, PG2 diisostearate, PG2 triisostearate, PG2 oleate, PG2 sesquioleate, PG2 dioleate, PG2 trioleate, PG3 caprylate, PG3 sesquicaprylate, PG3 dicaprylate, PG3 tricaprylate, PG3 caprate, PG3 sesquicaprate, PG3 dicaprate, PG3 tricaprate, PG3 laurate, PG3 sesquilaurate, PG3 dilaurate, PG3 trilaurate, PG3 myristate, PG3 sesquimyristate, PG3 dimyristate, PG3 trimyristate, PG3 stearate, PG3 sesquistearate, PG3 distearate, PG3 tristearate, PG3 isostearate, PG3 sesquiisostearate, PG3 diisostearate, PG3 triisostearate, PG3 oleate, PG3 sesquioleate, PG3 dioleate, PG3 trioleate, PG4 caprylate, PG4 sesquicaprylate, PG4 dicaprylate, PG4 tricaprylate, PG4 caprate, PG4 sesquicaprate, PG4 dicaprate, PG4 tricaprate, PG4 laurate, PG4 sesquilaurate, PG4 dilaurate, PG4 trilaurate, PG4 myristate, PG4 sesquimyristate, PG4 dimyristate, PG4 trimyristate, PG4 stearate, PG4 sesquistearate, PG4 distearate, PG4 tristearate, PG4 isostearate, PG4 sesquiisostearate, PG4 diisostearate, PG4 triisostearate, PG4 oleate, PG4 sesquioleate, PG4 dioleate, PG4 trioleate, PG5 caprylate, PG5 sesquicaprylate, PG5 dicaprylate, PG5 tricaprylate, PG5 tetracaprylate, PG5 caprate, PG5 sesquicaprate, PG5 dicaprate, PG5 tricaprate, PG5 tetracaprate, PG5 laurate, PG5 sesquilaurate, PG5 dilaurate, PG5 trilaurate, PG5 tetralaurate, PG5 myristate, PG5 sesquimyristate, PG5 dimyristate, PG5 trimyristate, PG5 tetramyristate, PG5 stearate, PG5 sesquistearate, PG5 distearate, PG5 tristearate, PG5 tetrastearate, PG5 isostearate, PG5 sesquiisostearate, PG5 diisostearate, PG5 triisostearate, PG5 tetraisostearate, PG5 oleate, PG5 sesquioleate, PG5 dioleate, PG5 trioleate, PG5 tetraoleate, PG6 caprylate, PG6 sesquicaprylate, PG6 dicaprylate, PG6 tricaprylate, PG6 tetracaprylate, PG6 pentacaprylate, PG6 caprate, PG6 sesquicaprate, PG6 dicaprate, PG6 tricaprate, PG6 tetracaprate, PG6 pentacaprate, PG6 laurate, PG6 sesquilaurate, PG6 dilaurate, PG6 trilaurate, PG6 tetralaurate, PG6 pentalaurate, PG6 myristate, PG6 sesquimyristate, PG6 dimyristate, PG6 trimyristate, PG6 tetramyristate, PG6 pentamyristate, PG6 stearate, PG6 sesquistearate, PG6 distearate, PG6 tristearate, PG6 tetrastearate, PG6 pentastearate, PG6 isostearate, PG6 sesquiisostearate, PG6 diisostearate, PG6 triisostearate, PG6 tetraisostearate, PG6 pentaisostearate, PG6 oleate, PG6 sesquioleate, PG6 dioleate, PG6 trioleate, PG6 tetraoleate, PG6 pentaoleate, PG10 caprylate, PG10 sesquicaprylate, PG10 dicaprylate, PG10 tricaprylate, PG10 tetracaprylate, PG10 pentacaprylate, PG10 hexacaprylate, PG10 caprate, PG10 sesquicaprate, PG10 dicaprate, PG10 tricaprate, PG10 tetracaprate, PG10 pentacaprate, PG10 hexacaprate, PG10 laurate, PG10 sesquilaurate, PG10 dilaurate, PG10 trilaurate, PG10 tetralaurate, PG10 pentalaurate, PG10 hexalaurate, PG10 myristate, PG10 sesquimyristate, PG10 dimyristate, PG10 trimyristate, PG10 tetramyristate, PG10 pentamyristate, PG10 hexamyristate, PG10 stearate, PG10 sesquistearate, PG10 distearate, PG10 tristearate, PG10 tetrastearate, PG10 pentastearate, PG10 hexastearate, PG10 isostearate, PG10 sesquiisostearate, PG10 diisostearate, PG10 triisostearate, PG10 tetraisostearate, PG10 pentaisostearate, PG10 hexaisostearate, PG10 oleate, PG10 sesquioleate, PG10 dioleate, PG10 trioleate, PG10 tetraoleate, PG10 pentaoleate, and PG10 hexaoleate.

Examples of polyoxyethylenated fatty esters that may be mentioned include the adducts of ethylene oxide with esters of lauric acid, palmitic acid, stearic acid or behenic acid, and mixtures thereof, especially those containing from 9 to 100 oxyethylene units, such as PEG-9 to PEG-50 laurate (CTFA names: PEG-9 laurate to PEG-50 laurate); PEG-9 to PEG-50 palmitate (CTFA names: PEG-9 palmitate to PEG-50 palmitate); PEG-9 to PEG-50 stearate (CTFA names: PEG-9 stearate to PEG-50 stearate); PEG-9 to PEG-50 palmitostearate; PEG-9 to PEG-50 behenate (CTFA names: PEG-9 behenate to PEG-50 behenate); polyethylene glycol 100 EO monostearate (CTFA name: PEG-100 stearate); and mixtures thereof

According to one of the embodiments of the present invention, the nonionic surfactant may be selected from polyoxyalkylenated derivatives of esters of polyols with fatty acids with a saturated or unsaturated chain containing, for example, from 8 to 24 carbon atoms, preferably 12 to 22 carbon atoms, preferably containing from 10 to 200, and more preferably from 10 to 100 oxyalkylene units, such as polyoxyalkylenated glyceryl esters of a C₈-C₂₄, preferably C₁₂-C₂₂, fatty acid or acids, preferably containing from 10 to 200, and more preferably from 10 to 100 oxyalkylene units; sorbitol esters of a C₈-C₂₄, preferably C₁₂-C₂₂, fatty acid or acids and polyoxyalkylenated derivatives thereof, preferably containing from 10 to 200, and more preferably from 10 to 100 oxyalkylene units; sugar (sucrose, maltose, glucose, fructose, and/or alkylglycose) esters of a C₈-C₂₄, preferably C₁₂-C₂₂, fatty acid or acids and polyoxyalkylenated derivatives thereof, preferably containing from 10 to 200, and more preferably from 10 to 100 oxyalkylene units; ethers of fatty alcohols; ethers of sugar and a C₈-C₂₄, preferably C₁₂-C₂₂, fatty alcohol or alcohols; and mixtures thereof.

As polyoxyalkylenated glyceryl esters of fatty acids, mono-, di- or triester of fatty acids with a polyoxyalkylenated glycerol (mono-, di- or triester of fatty acids with a polyallcylene glycol ether of glycerol), preferably polyoxyethylenated glyceryl stearate (mono-, di- and/or tristearate), such as PEG-20 glyceryl stearate (mono-, di- and/or tristearate) can be cited.

Mixtures of these surfactants, such as for example the product containing glyceryl stearate and PEG-100 stearate, marketed under the name ARLACEL 165 by Uniqema, and the product containing glyceryl stearate (glyceryl mono- and distearate) and potassium stearate marketed under the name TEGIN by Goldschmidt (CTFA name: glyceryl stearate SE), can also be used.

The sorbitol esters of C₈-C₂₄ fatty acids and polyoxyalkylenated derivatives thereof can be selected from sorbitan palmitate, sorbitan isostearate, sorbitan trioleate and esters of fatty acids and alkoxylated sorbitan containing for example from 20 to 100 EO, such as for example sorbitan monostearate (CTFA name: sorbitan stearate), sold by the company ICI under the name Span 60, sorbitan monopalmitate (CTFA name: sorbitan palmitate), sold by the company ICI under the name Span 40, and sorbitan tristearate 20 EU (CTFA name: polysorbate 65), sold by the company ICI under the name Tween 65, polyethylene sorbitan trioleate (polysorbate 85) or the compounds marketed under the trade names Tween 20 or Tween 60 by Uniqema.

As esters of fatty acids and glucose or alkylglucose, glucose palmitate, alkylglucose sesquistearates such as methylglucose sesquistearate, alkylglucose palmitates such as methylglucose or ethylglucose palmitate, methylglucoside fatty esters, the mixed ester of methylglucoside and the mixture of oleic acid/hydroxystearic acid (CTFA name: Methyl glucose dioleate/hydroxystearate), the ester of methylglucoside and isostearic acid (CTFA name: Methyl glucose isostearate), the ester of methylglucoside and lauric acid (CTFA name: Methyl glucose laurate), the mixture of monoester and diester of methylglucoside and isostearic acid (CTFA name: Methyl glucose sesqui-isostearate), the mixture of monoester and diester of methylglucoside and stearic acid (CTFA name: Methyl glucose sesquistearate) and in particular the product marketed under the name Glucate SS by AMERCHOL, and mixtures thereof can be cited.

As ethoxylated ethers of fatty acids and glucose or alkylglucose, ethoxylated ethers of fatty acids and methylglucose, and in particular the polyethylene glycol ether of the diester of methylglucose and stearic acid with about 20 moles of ethylene oxide (CTFA name: PEG-20 methyl glucose distearate) such as the product marketed under the name Glucam E-20 distearate by AMERCHOL, the polyethylene glycol ether of the mixture of monoester and diester of methyl-glucose and stearic acid with about 20 moles of ethylene oxide (CTFA name: PEG-20 methyl glucose sesquistearate) and in particular the product marketed under the name Glucamate SSE-20 by AMERCHOL and that marketed under the name Grillocose PSE-20 by GOLDSCHMIDT, and mixtures thereof, can for example be cited.

As sucrose esters, saccharose palmito-stearate, saccharose stearate and saccharose monolaurate can for example be cited.

As sugar ethers, alkylpolyglucosides can be used, and for example decylglucoside such as the product marketed under the name MYDOL 10 by Kao Chemicals, the product marketed under the name PLANTAREN 2000 by Henkel, and the product marketed under the name ORAMIX NS 10 by Seppic, caprylyl/capryl glucoside such as the product marketed under the name ORAMIX CG 110 by Seppic or under the name LUTENSOL GD 70 by BASF, laurylglucoside such as the products marketed under the names PLANTAREN 1200 N and PLANTACARE 1200 by Henkel, coco-glucoside such as the product marketed under the name PLANTACARE 818/UP by Henkel, cetostearyl glucoside possibly mixed with cetostearyl alcohol, marketed for example under the name MONTANOV 68 by Seppic, under the name TEGO-CARE CG90 by Goldschmidt and under the name EMULGADE KE3302 by Henkel, arachidyl glucoside, for example in the form of the mixture of arachidyl and behenyl alcohols and arachidyl glucoside marketed under the name MONTANOV 202 by Seppic, cocoylethylglucoside, for example in the form of the mixture (35/65) with cetyl and stearyl alcohols, marketed under the name MONTANOV 82 by Seppic, and mixtures thereof can in particular be cited.

Mixtures of glycerides of alkoxylated plant oils such as mixtures of ethoxylated (200 EO) palm and copra (7 EO) glycerides can also be cited.

The (d) nonionic surfactant according to the present invention preferably contains an alkenyl or branched C₁₂-C₂₂ acyl chain such as an oleyl or isostearyl group. More preferably, the nonionic surfactant according to the present invention is PEG-20 glyceryl triisostearate.

According to one of the embodiments of the present invention, the nonionic surfactant may be selected from copolymers of ethylene oxide and of propylene oxide, in particular copolymers of the following formula:

HO(C₂H₄O)ₐ(C₃H₆O)_{b}(C₂H₄O)_{c}H

in which a, b and c are integers such that a+c ranges from 2 to 100 and b ranges from 14 to 60, and mixtures thereof.

According to one of the embodiments of the present invention, the nonionic surfactant may be selected from silicone surfactants. Non-limiting mention may be made of those disclosed in documents U.S. Pat. No. 5,364,633 and U.S. Pat. No. 5,411,744.

The HLB of the nonionic surfactant(s) may preferably be from 11 to 16, more preferably 12 to 15, and even more preferably 12 to 14. If two or more nonionic surfactants are used, the HLB value is determined by the weight average of the HLB values of all the nonionic surfactants. The HLB is the ratio between the hydrophilic part and the lipophilic part in the molecule. This term HLB is well known to those skilled in the art and is described in “The HLB system. A time-saving guide to emulsifier selection” (published by ICI Americas Inc., 1984).

It is preferable that the (d) nonionic surfactant be selected from alkylpolyglucosides, more preferably C₆-C₂₄ alkylpolyglucosides. As C₆-C₂₄ alkyl polyglucosides, use is preferably made of those containing an alkyl group comprising from 6 to 24 carbon atoms and preferably from 8 to 16 carbon atoms, and containing a glucoside group preferably comprising 1, 2 or 3 glucoside units.

As examples of alkylpolyglucosides, mention may be made of caprylyl/capryl glucoside, palmkernel/coco glucoside, cetearyl glucoside, decyl glucoside, lauryl glucoside, coco-glucoside, arachidyl glucoside, C₁₂-C₂₀ alkyl glucoside, C₁₀-C₁₆ alkyl glucoside, myristyl glucoside, myristoyl ethyl glucoside, methyl coco-glucoside, tallow ethyl glucoside, undecyl glucoside, octyldodecyl glucoside, isostearyl glucoside, lauroyl ethyl gludoside, cocoyl ethyl glucoside, caproyl ethyl glucoside, and butyl glucoside, but not limited thereto.

The amount of the (d) glycerin fatty acid ester is not limited, but may range from 0.01 to 10% by weight, preferably from 0.05 to 7% by weight, and more preferably from 0.1 to 5% by weight, relative to the total weight of the composition.

### Cationic Polymer

The composition according to the present invention comprises at least one cationic polymer. A single type of cationic polymer may be used, but two or more different types of cationic polymer may be used in combination.

It should be noted that, for the purposes of the present invention, the term “cationic polymer” denotes any polymer containing cationic groups and/or groups that may be ionized into cationic groups.

Such polymers may be chosen from those already known per se as improving the cosmetic properties of the hair, i.e., especially those described in patent application EP-A-337 354 and in French patents FR-2 270 846, 2 383 660, 2 598 611, 2 470 596 and 2 519 863.

The (e) cationic polymers that are preferred are chosen from those containing units comprising primary, secondary, tertiary and/or quaternary amine groups, which may either form part of the main polymer chain or may be borne by a side substituent directly attached thereto.

The (e) cationic polymers used generally have a number-average molecular mass of between approximately 500 and approximately 5*10⁶ and preferably between approximately 10³ and approximately 3*10⁶.

Among the (e) cationic polymers that may be mentioned more particularly are polymers of the polyamine, polyamino amide and polyquaternary ammonium type.

These are known products. They are described in particular in French patents 2 505 348 and 2 542 997. Among the said polymers, mention may be made of the following.

(1) homopolymers or copolymers derived from acrylic or methacrylic esters or amides and comprising at least one of the units of formula (I), (II), (III) or (IV) below:

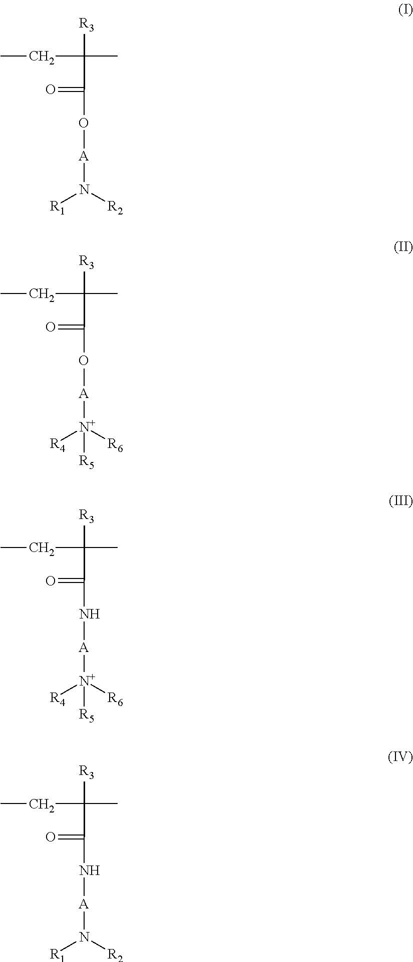

in which

R₃, which may be identical or different, denote a hydrogen atom or a CH₃ radical;

A, which may be identical or different, represent a linear or branched alkyl group of 1 to 6 carbon atoms, preferably 2 or 3 carbon atoms, or a hydroxyalkyl group of 1 to 4 carbon atoms;

R₄, R₅ and R₆, which may be identical or different, represent an alkyl group containing from 1 to 18 carbon atoms or a benzyl radical and preferably an alkyl group containing from 1 to 6 carbon atoms;

R₁ and R₂, which may be identical or different, represent hydrogen or an alkyl group containing from 1 to 6 carbon atoms, and preferably methyl or ethyl; and

X denotes an anion derived from an inorganic or organic acid, such as a methosulfate anion or a halide such as chloride or bromide.

The polymers of family (1) can also contain one or more units derived from comonomers which may be chosen from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinyl-caprolactam, and vinyl esters.

Thus, among these polymers of family (1), mention may be made of:
copolymers of acrylamide and of dimethylaminoethyl methacrylate quaternized with dimethyl sulfate or with a dimethyl halide, such as the product sold under the name Hercofloc by the company Hercules,
the copolymers of acrylamide and of methacryloyloxyethyltrimethylammonium chloride described, for example, in patent application EP-A-080 976 and sold under the name Bina Quat P 100 by the company BASF,
the copolymer of acrylamide and of methacryloyloxyethyltrimethylammonium methosulfate sold under the name Reten by the company Hercules,
quaternized or non-quaternized vinylpyrrolidone/dialkylaminoalkyl acrylate or methacrylate copolymers, such as the products sold under the name “Gafquat” by the company ISP, for instance “Gafquat 734” or “Gafquat 755”, or alternatively the products known as “Copolymer 845, 958 and 937”. These polymers are described in detail in French patents 2 077 143 and 2 393 573,
dimethylaminoethyl methacrylate/vinylcaprolactam/vinylpyrrolidone terpolymers, such as the product sold under the name Gaffix VC 713 by the company ISP, and
vinylpyrrolidone/methacrylamidopropyldimethylamine copolymers sold in particular under the name Styleze CC 10 by ISP, and quaternized vinylpyrrolidone/dimethylaminopropyl methacrylamide copolymers such as the product sold under the name “Gafquat HS 100” by the company ISP.

(2) The cellulose ether derivatives comprising quaternary ammonium groups, which are described in French patent 1 492 597, and in particular the polymers sold under the names “JR” (JR 400, JR 125, JR 30M) or “LR” (LR 400, LR 30M) by the company Amerchol. These polymers are also defined in the CTFA dictionary as hydroxyethylcellulose quaternary ammoniums that have reacted with an epoxide substituted with a trimethylammonium group.

(3) Cationic cellulose derivatives such as the copolymers of cellulose or cellulose derivatives grafted with a water-soluble quaternary ammonium monomer, described especially in U.S. Pat. No. 4,131,576, such as hydroxyalkylcelluloses, for instance hydroxymethyl-, hydroxyethyl- or hydroxypropylcelluloses grafted especially with a methacryloylethyltrimethylammonium, methacrylamidopropyltrimethylammonium or dimethyldiallylammonium salt.

The commercial products corresponding to this definition are more particularly the products sold under the name Celquat L 200 and Celquat H 100 by the company Akzo Nobel.

(4) The cationic guar gums described more particularly in U.S. Pat. Nos. 3,589,578 and 4,031,307, such as guar gums containing trialkylammonium cationic groups. Use is made, for example, of guar gums modified with a salt (e.g., chloride) of 2,3-epoxypropyltrimethylammonium. Mention may be made of guar hydroxypropyltrimonium chloride and hydroxypropyl guar hydroxypropyl trimonium chloride, such as those sold especially under the trade names Jaguar C13S, Jaguar Cl4S, Jaguar C17 and Jaguar C162 by the company Solvay.

(5) Polymers consisting of piperazinyl units and of divalent alkylene or hydroxyalkylene radicals containing straight or branched chains, optionally interrupted by oxygen, sulfur or nitrogen atoms or by aromatic or heterocyclic rings, and also the oxidation and/or quaternization products of these polymers. Such polymers are described, in particular, in French patents 2 162 025 and 2 280 361.

(6) Water-soluble polyamino amides prepared in particular by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyamino amides can be alkylated or, if they contain one or more tertiary amine functions, they can be quaternized. Such polymers are described, in particular, in French patents 2 252 840 and 2 368 508.

(7) Cyclopolymers of alkyldiallylamine or of dialkyldiallylammonium, such as the homopolymers or copolymers containing, as a main constituent of the chain, units corresponding to formula (V) or (VI):

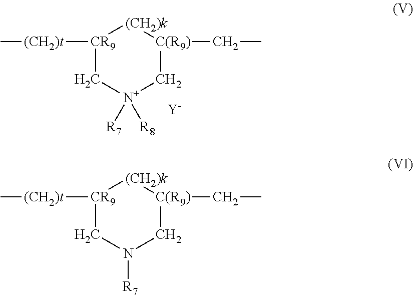

in which formulae

k and t are equal to 0 or 1, the sum k+t being equal to 1; R₉ denotes a hydrogen atom or a methyl radical; R₇ and R₈, independently of each other, denote an alkyl group having from 1 to 6 carbon atoms, a hydroxyalkyl group in which the alkyl group preferably has 1 to 5 carbon atoms, a lower (C₁-C₄) amidoalkyl group, or R₇ and R₈ can denote, together with the nitrogen atom to which they are attached, heterocyclic groups such as piperidyl or morpholinyl; R₇ and R₈, independently of each other, preferably denote an alkyl group having from 1 to 4 carbon atoms; and Y⁻ is an anion such as bromide, chloride, acetate, borate, citrate, tartrate, bisulfate, bisulfite, sulfate or phosphate. These polymers are described in particular in French patent 2 080 759 and in its Certificate of Addition 2 190 406.

Among the polymers defined above, mention may be made more particularly of the dimethyldiallylammonium chloride homopolymer sold under the name “Merquat 100” by the company Nalco (and its homologues of low weight-average molecular mass) and the copolymers of diallyldimethylammonium chloride and of acrylamide, sold under the name “Merquat 550”.

(8) The quaternary diammonium polymer containing repeating units corresponding to the formula:

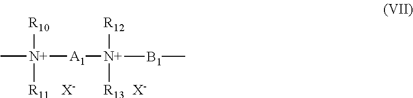

in which formula (VII):

R₁₀, R₁₁, R₁₂ and R₁₃, which may be identical or different, represent aliphatic, alicyclic or arylaliphatic radicals containing from 1 to 20 carbon atoms or lower hydroxyalkylaliphatic radicals, or alternatively R₁₀, R₁₁, R₁₂ and R₁₃, together or separately, constitute, with the nitrogen atoms to which they are attached, heterocycles optionally containing a second hetero atom other than nitrogen, or alternatively R₁₀, R₁₁, R₁₂ and R₁₃ represent a linear or branched C₁-C₆ alkyl radical substituted with a nitrile, ester, acyl or amide group or a group —CO—O—R₁₄-D or —CO—NH—R₁₄-D where R₁₄ is an alkylene and D is a quaternary ammonium group; A₁ and B₁ represent polymethylene groups containing from 2 to 20 carbon atoms which may be linear or branched, saturated or unsaturated, and which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and

X⁻ denotes an anion derived from an inorganic or organic acid;

A₁, R₁₀ and R₁₂ can form, with the two nitrogen atoms to which they are attached, a piperazine ring; in addition, if A₁ denotes a linear or branched, saturated or unsaturated alkylene or hydroxyalkylene radical, B₁ can also denote a group —(CH₂)ₙ—CO-D-OC—(CH₂)ₙ— in which D denotes:

i) a glycol residue of formula: —O—Z—O—, where Z denotes a linear or branched hydrocarbon-based radical or a group corresponding to one of the following formulae:

—(CH₂—CH₂13 O)ₓ—CH₂—CH₂—; and

—[CH₂—CH(CH₃)—O]_{y}—CH₂—CH(CH₃)13

where x and y denote an integer from 1 to 4, representing a defined and unique degree of polymerization or any number from 1 to 4 representing an average degree of polymerization;

ii) a bis-secondary diamine residue such as a piperazine derivative;

iii) a bis-primary diamine residue of formula —NH—Y—NH—, where Y denotes a linear or branched hydrocarbon-based radical, or alternatively the divalent radical CH₂—CH₂—S—S—CH₂—CH₂—; or

iv) a ureylene group of formula —NH—CO—NH—.

Preferably, X⁻ is an anion such as chloride or bromide.

These polymers generally have a number-average molecular mass of between 1000 and 100 000.

Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is more particularly possible to use polymers that consist of repeating units corresponding to the following formula (VIII):

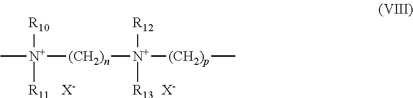

in which R₁₀, R₁₁, R₁₂ and R₁₃, which may be identical or different, denote an alkyl or hydroxyalkyl radical containing from 1 to 4 carbon atoms approximately, n and p are integers ranging from 2 to 20 approximately, and X⁻ is an anion derived from a mineral or organic acid.

(9) Polyamines such as Polyquart H sold by Cognis, which is given under the reference name “Polyethylene glycol (15) tallow polyamine” in the CTFA dictionary.

(10) Crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, in particular methylenebisacrylamide. A crosslinked acrylamide/methacryloyloxyethyltrimethylammonium chloride copolymer (20/80 by weight) in the form of a dispersion containing 50% by weight of the said copolymer in mineral oil can be used more particularly. This dispersion is sold under the name “Salcare® SC 92” by the company BASF. A crosslinked methacryloyloxyethyltrimethylammonium chloride homopolymer containing about 50% by weight of the homopolymer in mineral oil or in a liquid ester can also be used. These dispersions are sold under the names “Salcare® SC 95” and “Salcare® SC 96” by the company Allied Colloids.

(11) Other cationic polymers which can be used in the context of the present invention are polyalkyleneimines, in particular polyethyleneimines, polymers containing vinylpyridine or vinylpyridinium units, condensates of polyamines and of epichlorohydrin, quaternary polyureylenes and chitin derivatives.

It is preferable that the cationic polymer be a polyquaternium polymer or a polymeric quaternary ammonium salt.

Polymeric quaternary ammonium salts are cationic polymers comprising at least one quaternized nitrogen atom. Mention may in particular be made, as polymeric quaternary ammonium salts, of the Polyquaternium products (CTFA name), which contribute mainly to the quality of foam and feeling of the skin after use, in particular the feeling of the skin after use. These polymers can preferably be chosen from the following polymers:

Polyquaternium-5, such as the product Merquat 5 sold by Nalco;

Polyquatemium-6, such as the product Salcare SC 30 sold by BASF and the product Merquat 100 sold by Nalco;

Polyquaternium-7, such as the products Merquat S, Merquat 2200, Merquat 7SPR, and Merquat 550 sold by Nalco and the product Salcare SC 10 sold by BASF;

Polyquaternium-10, such as the product Polymer JR400 sold by Amerchol;

Polyquaternium-11, such as the products Gafquat 755, Gafquat 755N and Gafquat 734 sold by ISP;

Polyquaternium-15, such as the product Rohagit KF 720 F sold by Rohm;

Polyquaternium-16, such as the products Luviquat FC905, Luviquat FC370, Luviquat HM552 and Luviquat FC550 sold by BASF;

Polyquaternium-28, such as the product Styleze CC10 sold by ISP;

Polyquaternium-44, such as the product Luviquat Care sold by BASF;

Polyquaternium-46, such as the product Luviquat Hold sold by BASF; and

Polyquaternium-47, such as the product Merquat 2001 sold by Nalco.

Preferably, the cationic polymer is chosen from, Polyquaternium-10, Polyquaternium-47, hydroxypropyl guar hydroxypropyl trimonium chloride and their mixtures.

The amount of the (e) cationic polymer is not limited, but the amount of the cationic polymer may be from 0.01 to 10% by weight, preferably 0.05 to 5% by weight, and more preferably 0.1 to 1% by weight, relative to the total weight of the composition.

### Water

The composition according to the present invention may comprise (f) water.

The amount of (f) water is not limited, and may be from 40 to 95% by weight, preferably from 45 to 90% by weight, and more preferably 50 to 85% by weight, relative to the total weight of the composition.

### Other Ingredients

The composition according to the present invention may also comprise an effective amount of other ingredients, known previously elsewhere in cosmetic compositions, such as various common adjuvants, oils such as ester oils and silicone oils, sequestering agents such as EDTA and etidronic acid, preserving agents, vitamins or provitamins, for instance, panthenol, opacifiers, fragrances, plant extracts, and so on.

The composition according to the present invention may further comprise at least one organic solvent. So the organic solvent is preferably water miscible. As the organic solvent, there may be mentioned, for example, C₁-C₄ alkanols, such as ethanol and isopropanol; aromatic alcohols such as benzyl alcohol and phenoxyethanol; analogous products; and mixtures thereof.

The organic water-soluble solvents may be present in an amount ranging from less than 10% by weight, preferably from 5% by weight or less, and more preferably from 1% by weight or less, relative to the total weight of the composition.

If necessary, the composition according to the present invention may comprise a small amount of alkylethersulfate(s) and/or alkylsulfate(s) so far as the desired properties of the composition of the present invention can be maintained. It is preferable that the composition according to the present invention not comprise any substantial amount of alkylsulfates such as SLS (sodium lauryl sulfate) or alkylethersulfates such as SLES (sodium lauryl ether sulfate). Thus, it is preferable that the composition according to the present invention comprise 1% by weight or less, preferably 0.1% by weight or less, and more preferably 0.01% by weight or less of alkylsulfates or alkylethersulfates, relative to the total weight of the composition. It is most preferable that the composition according to the present invention comprise no alkylsulfate or alkylethersulfate.

### Preparation

The composition according to the present invention can be prepared by mixing the above essential and optional ingredients in accordance with a conventional process. The conventional process includes mixing with a high pressure homogenizer (a high energy process).

### Process and Use

It is preferable that the composition according to the present invention be a cosmetic composition, preferably a cleansing composition, and more preferably a cleansing composition for keratin fibers.

The cosmetic composition according to the present invention can constitute, in particular, foaming creams for topical application used, in particular, in the cosmetic field as products for cleaning or removing make-up from the skin (body or face, including the eyes), scalp and/or hair. They can constitute, more particularly, a composition for cleansing keratin fibers, in particular hair.

The composition according to the present invention can be used for a non-therapeutic process, such as a cosmetic process, for treating the skin, the hair, and/or the scalp, by being applied to the skin, the hair, and/or the scalp.

The present invention may also relate to a use of the composition according to the present invention for the manufacture of a cleansing composition, preferably a shampoo, with no irritation or reduced irritation to the scalp.

### EXAMPLES

The present invention will be described in more detail by way of examples, which however should not be construed as limiting the scope of the present invention.

### Examples 1-4 and Comparative Examples 1-3

The following compositions according to Examples 1-4 and Comparative Examples 1-3, shown in Table 1, were prepared by mixing the components shown in Table 1. The numerical values for the amounts of the ingredients shown in Table 1 are all based on “% by weight” as active raw materials.

TABLE 1

| | | | | | Comp. | Comp. | Comp. |
|---|---|---|---|---|---|---|---|
| Ingredients | Ex. 1 | Ex. 2 | Ex. 3 | Ex. 4 | Ex. 1 | Ex. 2 | Ex. 3 |
| Sodium C14-16 Olefin | 9.2 | 9.2 | 9.2 | 9.2 | 9.2 | 9.2 | — |
| Sufonate | | | | | | | |
| Sodium Laureth Sulfate | — | — | — | — | — | — | 9.2 |
| Coco-betaine | 2.5 | 7 | 8 | 10 | 2.5 | 2.5 | 2.5 |
| Coco-glucoside | 2.5 | 2.5 | 2.5 | 2.5 | 2.5 | 2.5 | 2.5 |
| Glyceryl Caprylate | 0.5 | 0.5 | 0.5 | 0.5 | — | — | — |
| Cocamide MBA | — | — | — | — | — | 0.5 | — |
| Polyquaternium-10 | 0.5 | 0.5 | 0.5 | 0.5 | 0.5 | 0.5 | 0.5 |
| Dimethicone | 1.75 | 1.75 | 1.75 | 1.75 | 1.75 | 1.75 | 1.75 |
| Carbomer | 0.25 | 0.25 | 0.25 | 0.25 | 0.25 | 0.25 | 0.25 |
| Glycol Distearate | 1.2 | 1.2 | 1.2 | 1.2 | 1.2 | 1.2 | 1.2 |
| Sodium Benzoate | 0.5 | 0.5 | 0.5 | 0.5 | 0.5 | 0.5 | 0.5 |
| Salicylic Acid | 0.2 | 0.2 | 0.2 | 0.2 | 0.2 | 0.2 | 0.2 |
| Fragrance | 0.5 | 0.5 | 0.5 | 0.5 | 0.5 | 0.5 | 0.5 |
| Sodium Chloride | qs | qs | qs | qs | qs | qs | qs |
| Hexylene Glycol | qs | qs | qs | qs | qs | qs | qs |
| Sodium Hydroxide | qs | qs | qs | qs | qs | qs | qs |
| Citric Acid | qs | qs | qs | qs | qs | qs | qs |
| Water | qs 100 | qs 100 | qs 100 | qs 100 | qs 100 | qs 100 | qs 100 |
| Amount of Foam (ml) | 503 | 453 | 449 | 437 | 405 | 416 | 348 |

qs: quantum sufficiat (appropriate amount)
qs 100: appropriate amount up until 100% by weight (balance)

### Evaluation

(Amount of Foam)

The amount of foam produced by each of the compositions according to Examples 1-4 and Comparative Examples 1-3 was measured by a so-called “blender method”.

A sample was prepared by dissolving 4 g of the composition into 76 g of water with a hardness of 10 fH to prepare a 5% aqueous shampoo liquid. The sample was agitated by a blender (HR2003, PHILIPS) for 1 minute. Agitation speed was around 10,000 rpm. The volume of the foam generated by the agitation was measured.

The results are shown in Table 1.

It is clear from the results of Examples 1-4 and Comparative Example 3 shown in Table 1 that the use of linear olefin sulfonate (sodium C14-16 olefin sulfonate) can increase the volume of foam, as compared to the use of alkylethersulfate (sodium laureth sulfate).

It is also clear from the results of Examples 1-4 and Comparative Examples 1 and 2 shown in Table 1 that the use of glycerin fatty acid ester (glyceryl caprylate) can contribute to the further increase of foam volume, as compared to no use of the glycerin fatty acid ester (Comparative Example 1) and the use of the conventional nonionic surfactant (cocamide MEA).

In comparison with Examples 1-4, it can be understood that it is preferable for the amount of amphoteric surfactant (coco-betaine) to be smaller (e.g., 10% by weight or less) in terms of foam volume.

(Storage Stability)

The stability of each of the compositions according to Examples 1-4 and Comparative Example 2 was evaluated visually, after storing the compositions in incubators (Yamato IC800) at 45° C. for 12 weeks, under the following criteria.

Good: No separation was observed during the storage.

Fair: Separation was observed later.

Poor: Separation immediately after the start of the storage was observed.

The results are shown in Table 2.

TABLE 2

| | Ex. 1 | Ex. 2 | Ex. 3 | Ex. 4 | Comp. Ex. 2 |
|---|---|---|---|---|---|
| Storage | Good | Good | Fair | Fair | Good |
| Stability | | | | | |

It is clear from the results of Examples 1-4 and Comparative Example 2 shown in Table 2 that the storage stability may be improved even by the use of a conventional nonionic surfactant (cocamide MEA: Comparative Example 2) instead of glycerin fatty acid ester (glyceryl caprylate: Examples 1-4). However, as shown in Table 1, the foam quality deteriorates if the conventional nonionic surfactant is used.

In comparison with Examples 1-4, it can be understood that it is preferable for the amount of an amphoteric surfactant (coco-betaine) to be 7% by weight or less in terms of storage stability.

(Sensory Test)

The compositions according to Examples 1-4 cause no or only a little irritation in sensory tests by panelists. On the other hand, the composition according to Comparative Example 3 causes irritation.

1. A composition comprising:
(a) at least one linear olefin sulfonate;
(b) at least one glycerin fatty acid ester; and
(c) at least one amphoteric surfactant.

2. The composition according to claim 1, wherein the (a) linear olefin sulfonate is selected from linear C₁₂-C₁₈ olefin sulfonates.

3. The composition according to claim 1 or 2, wherein the amount of the (a) linear olefin sulfonate ranges from 1 to 20% by weight, preferably from 3 to 15% by weight, and more preferably from 5 to 10% by weight, relative to the total weight of the composition.

4. The composition according to any one of claims 1 to 3, wherein the (b) glycerin fatty acid ester is a monoester.

5. The composition according to any one of claims 1 to 4, wherein the (b) glycerin fatty acid ester is a monoester of glycerin and C₈-C₁₈ fatty acid, preferably C₈-C₁₄ fatty acid, and more preferably C₈-C₁₂ fatty acid, preferably selected from the group consisting of glyceryl caprylate, glyceryl caprate, glyceryl laurate, glyceryl myristate, glyceryl stearate, glyceryl isostearate and a mixture thereof.

6. The composition according to any one of claims 1 to 5, wherein the amount of the (b) glycerin fatty acid ester ranges from 0.01 to 10% by weight, preferably from 0.05 to 5% by weight, and more preferably from 0.1 to 1% by weight, relative to the total weight of the composition.

7. The composition according to any one of claims 1 to 6, wherein the (c) amphoteric surfactant is selected from betaine-type surfactants.

8. The composition according to any one of claims 1 to 7, wherein the amount of the (c) amphoteric surfactant ranges from 0.01 to 10% by weight, preferably from 0.05 to 7% by weight, and more preferably from 0.1 to 5% by weight, relative to the total weight of the composition.

9. The composition according to any one of claims 1 to 8, wherein the composition further comprises (d) at least one nonionic surfactant, preferably alkylpolyglucoside.

10. The composition according to claim 9, wherein the amount of the (d) nonionic surfactant ranges from 0.01 to 10% by weight, preferably from 0.05 to 7% by weight, and more preferably from 0.1 to 5% by weight, relative to the total weight of the composition.

11. The composition according to any one of claims 1 to 10, wherein the composition further comprises (e) at least one cationic polymer.

12. The composition according to any one of claims 1 to 11, wherein the composition comprises 1% by weight or less, preferably 0.1% by weight or less, and more preferably 0.01% by weight or less of alkylsulfates or alkylethersulfates, relative to the total weight of the composition.

13. The composition according to any one of claims 1 to 12, wherein the composition is intended for a cosmetic composition, preferably a cleansing composition, and more preferably a cleansing composition for keratin fibers.

14. Use of the composition according to any one of claims 1 to 13, for the manufacture of a cleansing composition, preferably a shampoo, with no irritation or reduced irritation to the scalp.

15. A cosmetic process for treating the skin, the hair, and/or the scalp, comprising the step of applying the composition according to any one of claims 1 to 14 to the skin, the hair, and/or the scalp.
